(19) United States
(12) Patent Application Publication
(10) Pub. No.: US 2015/0273120 A1
(43) Pub. Date: Oct. 1, 2015

(54) MEDICAL DEVICES FOR DRAINAGE

(71) Applicant: Boston Scientific Scimed, Inc., Maple Grove, MN (US)

(72) Inventors: Nathan Zamarripa, Sudbury, MA (US); Jeff Gray, Sudbury, MA (US); Alison Lepordo, North Andover, MA (US)

(21) Appl. No.: 14/668,437

(22) Filed: Mar. 25, 2015

Related U.S. Application Data
(60) Provisional application No. 61/970,694, filed on Mar. 26, 2014.

(51) Int. Cl. A61M 1/00

(52) U.S. Cl. CPC A61M 1/008

(57) ABSTRACT

A medical device for drainage of fluids is disclosed. The medical device may be used for drainage of urine from the renal pelvis to the bladder. The medical device includes an elongate shaft having a proximal end, a distal end, a first lumen, and a second lumen. A first inflatable balloon is positioned proximal to the distal end of the elongate shaft and in fluid communication with the first lumen. At least one aperture is positioned adjacent to the inflatable balloon and in fluid communication with the second lumen.

### CROSS-REFERENCE TO RELATED APPLICATIONS

This application claims the benefit of U.S. Provisional Application No. 61/970,694, filed Mar. 26, 2014, the entire content of which is incorporated herein by reference.

### TECHNICAL FIELD The present disclosure generally relates to medical devices and more particularly to balloon stents and catheters for drainage of fluids from a patient's body.

### BACKGROUND

A wide variety of intracorporeal medical devices have been developed for medical use. Some of these devices include stents, guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

An example medical device may include a medical device for drainage of fluid within the body, for example, urine from the renal pelvis to the bladder. An example medical device includes an elongate shaft, a first inflatable balloon and at least one aperture. The elongate shaft has a proximal end, a distal end, a first lumen, and a second lumen. The first inflatable balloon is positioned proximal to the distal end of the elongate shaft and in fluid communication with the first lumen. The at least one aperture is positioned adjacent to the inflatable balloon and in fluid communication with the second lumen.

In another example, the medical device includes an elongate shaft, a hub assembly, a first inflatable balloon, a second inflatable balloon, and a plurality of holes. The elongate shaft has a proximal end, a distal end, a first lumen and a second lumen.

The hub assembly is affixed adjacent to the proximal end of the elongate shaft. The hub assembly includes a Luer valve and a drainage port. The first inflatable balloon is in fluid communication with the first lumen. The second inflatable balloon is in fluid communication with the first lumen. A plurality of holes is in fluid communication with the second lumen.

Still another example medical device includes an elongate shaft, a hub assembly, a first inflatable balloon, a second inflatable balloon, a first plurality of holes, and a second plurality of holes. The elongate shaft has a proximal end, a distal end, a first lumen, and a second lumen. The hub assembly is affixed adjacent to the proximal end of the elongate shaft. The hub assembly includes a Luer valve and a drainage port. The first inflatable balloon is positioned adjacent to the distal end and in fluid communication with the first lumen. The second inflatable balloon is positioned proximal to the first inflatable balloon and in fluid communication with the first lumen. The first plurality of holes is positioned distal to the first inflatable balloon and in fluid communication with the second lumen. The second plurality of holes is positioned proximal to the second inflatable balloon and in fluid communication with the second lumen.

The above summary of some example embodiments is not intended to describe each disclosed embodiment or every implementation of the disclosed subject matter. The Figures, and Detailed Description, which follow, more particularly exemplify these embodiments

### BRIEF DESCRIPTION OF THE DRAWINGS

The disclosed subject matter may be more completely understood in consideration of the following detailed description of various embodiments in connection with the accompanying drawings, in which:

FIG. 1 is a schematic side view illustrating an exemplary medical device.

FIG. 2 is a schematic side view of the illustrative medical device of FIG. 1 in an inflated state.

FIG. 3 is a cross-sectional view of the illustrative device of FIG. 2, taken at line 3-3.

FIG. 4 is a schematic view illustrating the use of the medical device of FIG. 1 in the urinary system.

FIG. 5 is a schematic view illustrating another illustrative medical device with one inflatable balloon.

While the disclosed subject matter is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

The following description should be read with reference to the drawings, wherein like reference numerals indicate like elements throughout the several views. The drawings, which are not necessarily to scale, are not intended to limit the scope of the claimed invention. The detailed description and drawings illustrate exemplary embodiments of the claimed invention. Those skilled in the art will recognize that the various elements described and/or shown may be arranged in various combinations and configurations without departing from the scope of the disclosure. The detailed description and drawings illustrate example embodiments of the claimed invention.

All numbers or values are herein assumed to be modified by the term “about.” The disclosure of numerical ranges by endpoints includes all numbers subsumed within that range (e.g., 1 to 5 includes 1, 1.5, 2, 2.75, 3, 3.80, 4, and 5).

As used in this specification and the appended claims, the singular indefinite articles “a”, “an”, and the definite article “the” should be considered to include or otherwise cover both single and plural referents unless the content clearly dictates otherwise. In other words, these articles are applicable to one or more referents. As used in this specification and the appended claims, the term “or” is generally employed to include or otherwise cover “and/or” unless the content clearly dictates otherwise.

References in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases do not necessarily refer to the same embodiment. Further, any particular feature, structure, or characteristic described in connection with a particular embodiment is intended to be applied, incorporated or substituted into other embodiments, whether or not explicitly described, unless clearly stated to the contrary.

Certain medical treatments are aimed at removing or transporting excess fluids from various parts of the body. In some instances, ureteral stents may be used to create a pathway for urinary drainage from the kidney to the bladder in patients with ureteral obstruction or injury. Ureteral stents may also be used to enhance or otherwise protect the integrity of the ureter in a variety of surgical applications. A number of clinical conditions can produce interruption in urine flow including, for example, intrinsic obstruction of the ureter due to tumor growth, stricture or stones, compression of the ureter due to extrinsic tumor growth, stone fragment impaction in the ureter following extracorporeal shock wave lithotripsy, and ureteral procedures such as endopyelotomy and ureteroscopy. Stents may be used to treat or avoid obstructions of the ureter (such as ureteral stones or ureteral tumors) that disrupt the flow of urine from the corresponding kidney to the bladder. Serious obstructions of the urinary tract may cause urine to back up into the kidney, thereby threatening renal function. Ureteral stents may also be used after endoscopic inspection of the ureter.

In one application, a ureteral stent may assist in the flow of urine from the kidney to the bladder. The region referred to as the ureterovesical junction is a small area of the ureter that is immediately upstream, relative to normal urine flow, of the bladder. The ureterovesicle junction may be more sensitive, for example, may cause a greater pain sensation, relative to other regions of the ureter wall and kidneys, and therefore may be a source, in some cases a significant source, of patient discomfort when this region of the ureter contacts indwelling ureteral stents.

Some ureteral stents or catheters may include one or more pigtail loops for securing these devices in place. The pigtail loops may retain the stents or catheters due to its irregular pigtail shape or with the help of additional fixation mechanisms such as sutures. However, the pigtail loops may have a sharp tip and irregular shape that can cause damage to surrounding tissue and complications in its fixation and removal. In other instances, the location and geometry of the pigtail loops may cause discomfort to the patient. In some other instances, the space occupied by the pigtail loops inside the kidney may reduce the space available for drainage.

Some embodiments of the present disclosure relate to drainage of excess fluid, such as urine, in order to achieve a therapeutic effect. For example, the devices and methods described herein may be used for draining urine from the renal pelvis to the bladder with or without external access to the stent for fluid management. While many of the devices and methods described herein are discussed relative to drainage of urine from a patient's kidney, it is contemplated that the devices and methods may be used in other treatment locations and/or applications where drainage of excess fluids (for example, urine, bile, post-operative fluids) is desired.

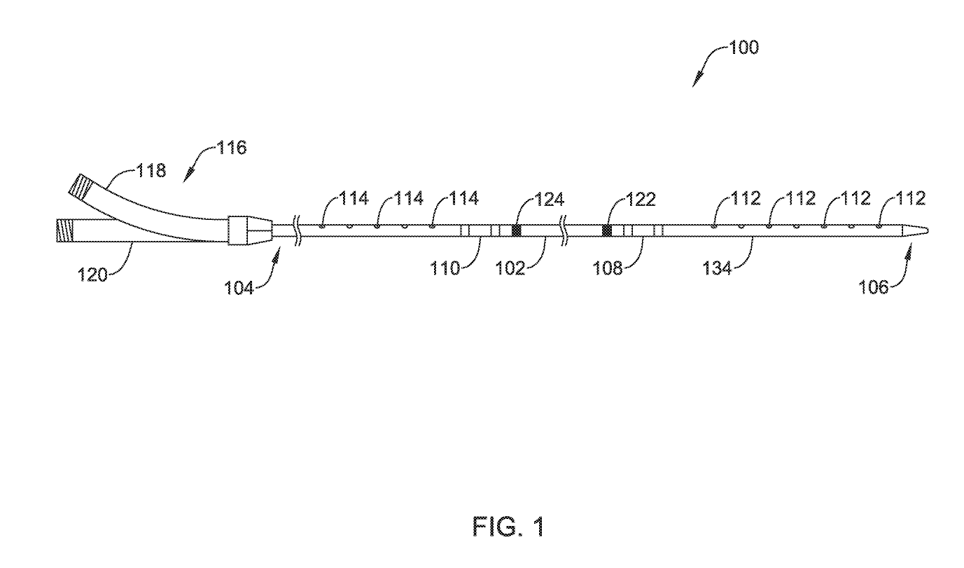

Turning to FIG. 1, an illustrative medical device 100 for providing drainage is shown. The device 100 may be configured to drain fluids such as urine, bile juice, pus, blood, and/or other fluids from patient's body. The medical device 100 may include an elongate shaft 102 extending from a proximal end 104 to a distal end 106. The device 100 may include a hub assembly 116 configured to remain outside of the body positioned adjacent to the proximal end 104 of the elongate shaft 102. In some instances, the hub assembly 116 may include a drainage and/or flush port 118 and/or a Luer valve 120. It is contemplated that the hub assembly 116 may include fewer than two ports or more than two ports as desired. For example, the hub assembly 116 may include one, two, three, four, or more ports as desired.

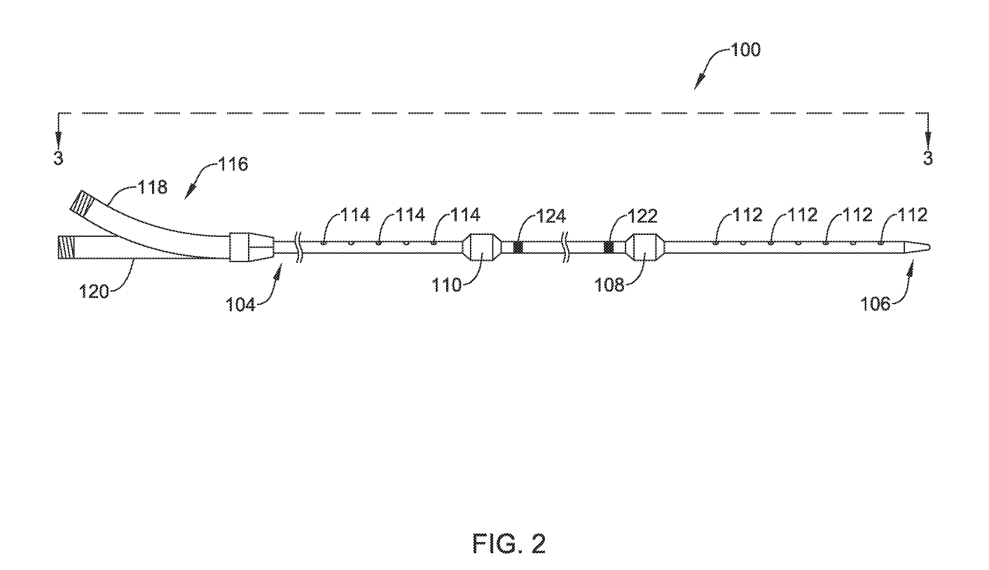

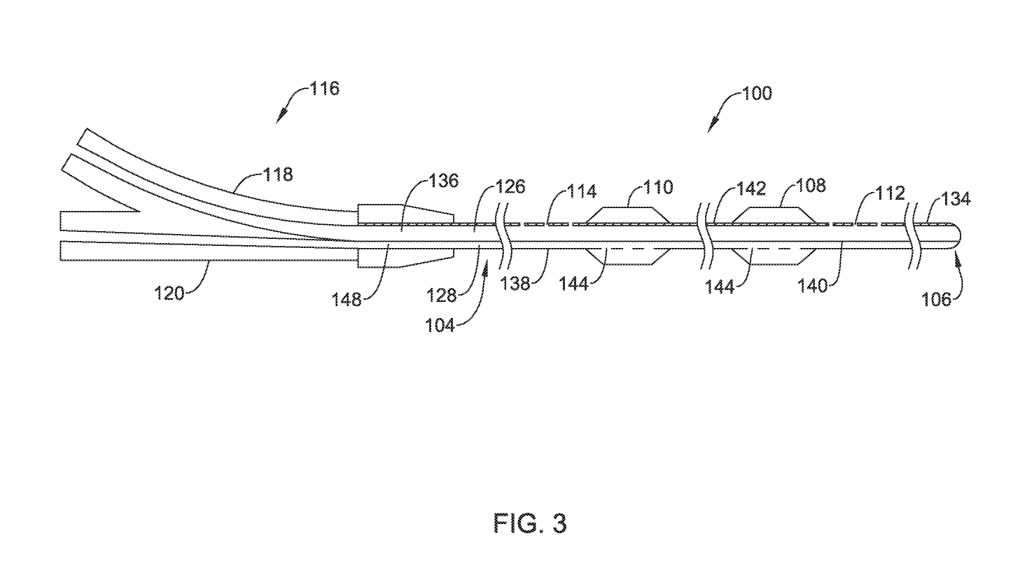

In some embodiments, the medical device 100 may include a first inflatable balloon 108 positioned proximal to the distal end 106 of the elongate shaft 102. The first inflatable balloon 108 may be referred to as the first balloon 108 hereinafter. The first balloon 108 may be in fluid communication a first lumen or an inflation lumen 128 (shown in FIG. 3) which is in fluid communication with the Luer valve 120 as will be discussed in more detail with respect to FIG. 3. In some embodiments, a second inflatable balloon 110 may be positioned proximal to the first balloon 108 and may also be in fluid communication with the inflation lumen 128. The balloons 108, 110 shown in FIG. 1 are in a deflated or compressed state. The balloons 108, 110 may be kept in the deflated state during insertion to the target location. Once the device 100 has been inserted to the target location, the balloons 108, 110 may be inflated, as shown in FIG. 2, to secure the device 100 at the target location. In some embodiments, there may be fewer than two or additional inflatable balloons disposed on the elongate shaft 102 that may be helpful for positioning or treatment. For example, there may one, two, three, four, or more balloons disposed on the elongate shaft 102. In some embodiments, the length of the inflated balloons 108, 110 may be in the range of from about 1 centimeter (cm) to 3 cm. This is just an example. It is contemplated that the balloons 108, 110 may have a length shorter than 1 cm or greater than 3 cm as desired for a given application.

While not explicitly shown, in some embodiments, the balloons 108, 110 may include a one-way valve such that once the balloons 108, 110 are inflated, user intervention is required to deflate the balloons 108, 110. In other embodiments, a one-way valve may be placed at other locations in the device 100. For example, in some instances, a one-way valve may be placed in the inflation lumen 128 or in the hub assembly 116. It is contemplated that the balloons 108, 110 may be partially inflated to different degrees to achieve sufficient fixation without damaging tissue or causing patient discomfort. In some instances, the first and second balloons 108, 110 may be in fluid communication with two separate inflation lumens such that the inflation of each of the balloons 108, 110 may be separately controlled.

The medical device 100 may include at least one aperture or hole 112, 114 extending from an outer surface 134 of the elongate shaft 102 to a second lumen or drainage lumen 126 positioned within the elongate shaft 102. In some instances, a first plurality of apertures 112 (also referred to as “holes”) may be positioned adjacent to and distal to the first balloon 108 and in fluid communication with a drainage lumen 126 (shown in FIG. 3). While the first plurality of apertures 112 is illustrated as including seven apertures, it is completed that there may be fewer than or more than seven apertures. For example, there may be one, two, three, four, or more apertures 112 positioned distal to the first balloon 108. It is further contemplated that in some instances, the plurality of apertures 112 may be positioned adjacent to and proximal to the first balloon 108. In some instances, the device 100 may include a second plurality of apertures 114 positioned adjacent to and proximal to the second balloon 110 and in fluid communication with the drainage lumen 126. While the second plurality of apertures 114 is illustrated as including five apertures, it is completed that there may be fewer than or more than five apertures. For example, there may be one, two, three, four, or more apertures 114 positioned proximal to the second balloon 110. It is further contemplated that in some instances, the plurality of apertures 114 may be positioned adjacent to and distal to the second balloon 110. In some embodiments, one of the two pluralities of apertures 112, 114 may not be present. For example, in some instances, the device 100 may include only the first plurality of apertures 112 while in other instances, the device may include only the second plurality of apertures 114. Excess body fluids, such as, but not limited to, urine, may enter or exit the drainage lumen 126 via the apertures 112, 114 or holes. In some instances, urine from the bladder may enter the drainage lumen 126 through the first plurality of apertures 112 and exit into the kidney through the second plurality of apertures 114 due to concentration difference, gravity, or suction, etc. or through the drainage port 118. This is just an example.

In some embodiments, the medical device 100 may include one or more radiopaque markers 122, 124 disposed adjacent to the balloons 108 and 110 to facilitate placement of the device. The physician may identify the location of the balloons using X-ray technology during insertion or removal of the medical device 100 in the patient's body.

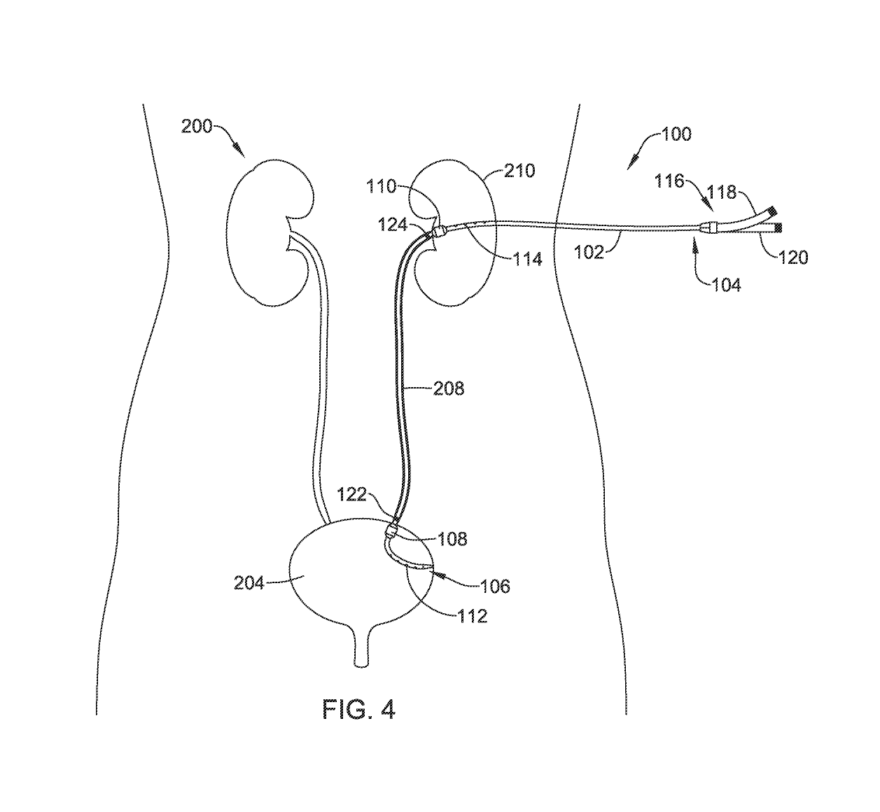

FIG. 2 illustrates a schematic side view of the medical device 100 including the balloons 108, 110 in an inflated state. The balloons 108, 110 may be configured to inflate by passing inflation media through the Luer valve 120 and through the inflation lumen 128. Inflation of the balloons 108, 110 may hold the balloons in a desired position, thereby eliminating need for pig tail loops or other mechanisms for securing the medical device in place for drainage. For example, as will be discussed in more detail with respect to FIG. 4, the first balloon 108 may be inflated within the bladder to position the first plurality of apertures 112 within the bladder. The second balloon 110 may be inflated within the kidney to prevent the device 100 from migrating further into the bladder. Excess fluid, such as urine, may enter the drainage lumen 126 through the first plurality of apertures 112 and exit through the second plurality of apertures 114 and/or the drainage port 118.

FIG. 3 illustrates a cross-sectional view of FIG. 2 taken at line 3-3. The elongate shaft 102 may include an outer tubular member 138 and an inner tubular member 140 forming a drainage lumen 126 and an inflation lumen 128. It is contemplated that the drainage lumen 126 and the inflation lumen 128 may be fluidly isolated from one another such that bodily fluids do not enter the inflation lumen 128 and inflation media does not enter the drainage lumen 126. The scale of FIG. 3 has changed from that of FIG. 2 in order to better illustrate the lumens 126, 128.

In some embodiments, the outer tubular member 138 and the inner tubular member 140 may be positioned such that the drainage lumen 126 and the inflation lumen 128 may extend side-by-side along the length of the elongate shaft 102. However, the lumens 126, 128 may be arranged in any manner desired, such as, but not limited to a co-axial or concentric arrangement. In some instances, a portion of the outer surface of the inner tubular member 140 may be joined with or extend along an inner surface of the outer tubular member 138 to form a common wall 142.

The first plurality of apertures 112 and the second plurality of apertures 114 may extend from the outer surface 134 of the elongate shaft 102, through the common wall, and into the drainage lumen 126 such that the drainage lumen 126 is in fluid communication with the outer surface 134 of the elongate shaft 102. The drainage lumen 126 may extend from the proximal end 104 of the elongate shaft 102 to the distal end 106 of the elongate shaft 102. The proximal end 136 of the drainage lumen 126 may be in fluid communication with the drainage and/or flush port 118. The drainage port 118 may be configured to drain, for example, urine from the bladder and/or kidney. In some instances, suction devices may be attached to or otherwise in communication with the drainage port 118 to facilitate evacuation of bodily fluids.

In some instances, the outer tubular member 138 may include one or more apertures 144 extending from the inflation lumen 128 to the interior of the inflation balloons 108, 110. Apertures 144 may allow inflation fluid to enter the balloons 108, 110. In some instances, apertures 144 may include or may be replaced by one or more one-way valves. It is further contemplated that separate inflation lumens may be provided to each of the balloons 108, 110, although this is not required. The inflation lumen 128 may extend from the proximal end 104 of the elongate shaft 102 to the distal end 106 of the elongate shaft 102. The proximal end 148 of the inflation lumen 128 may be in fluid communication with the Luer valve 120. The Luer valve 120 may be configured to receive inflation media to inflate the balloons 108, 110. In some instances, the Luer valve 120 may also be used to deflate the balloons 108, 110.

In some instances, the cross-sectional area of the drainage lumen 126 may be larger than the cross-sectional area of the inflation lumen 128, although this is not required. It is contemplated that in some embodiments, the drainage lumen 126 and the inflation lumen 128 may have similar cross-sectional areas. In other embodiments, the cross-sectional area of the inflation lumen 128 may be larger than the cross-sectional area of the drainage lumen 126.

For nephroureteral applications, FIG. 4 illustrates how the medical device 100 may be secured within a patient's urinary system 200. The medical device 100 may be implanted within a patient's body by any conventional method known in the art. For example, the device 100 may be delivered to the desired location percutaneously, as illustrated in FIG. 4. The device 100 may be advanced through the ureter 208 until the distal end 106 and the first balloon 108 are disposed within a portion of the bladder 204. Radiopaque marker 122 may facilitate placement of the device 100. When the first balloon 108 is positioned within the bladder 204, the second balloon 110 may be disposed within a portion of the kidney 210, such as the renal pelvis. This may position the first plurality of apertures 112 within the bladder 204 and the second plurality of apertures 114 within the kidney 210.

In some embodiments, as the inflation media is passed through the inflation lumen 128, the first balloon 108 inflates thereby anchoring the distal end 106 in the bladder 204. The second balloon 110 may also be inflated to anchor the device 100 within the kidney 210 and to prevent further distal movement of the device 100. In some embodiments, urine to be drained from the bladder 204 enters the drainage lumen 126 through the first plurality of apertures 112 and flows through the drainage lumen 126 towards the proximal end 104 of the elongate shaft 102. In some instances, some urine may exit the drainage lumen 126 and enter the kidney 210 while some urine may exit the device 100 through the drainage port 118. In some instances, urine to be drained from the kidney 210 may also enter the drainage lumen 126 through the second plurality of apertures 112 and flow through the drainage lumen 126 towards the proximal end 104 of the elongate shaft 102. It is contemplated that additional devices, such as suction devices, may be used to facilitate removal of urine from the urinary system.

It is contemplated that the smooth geometry of balloons 108, 110 may reduce or eliminate patient discomfort caused by irritation from the pigtail loops of previous devices. In addition, the smooth geometry and ability to control inflation may reduce or minimize damage to surrounding tissue during positioning, securing and removal. In some embodiments, the device 100 may be easy to fixate in position by the physician thereby avoiding use of additional fixation members like sutures. In some embodiments, inflation and deflation of the balloons 108, 110 may allow easy insertion and removal of the medical device 100 into and from the patient's body respectively.

In some other embodiments, the medical device 100 may be used to drain other fluids such as bile juice from bile duct to liver. In other embodiments, the medical device 100 may be used to drain fluids from any part of the patient's body, for example, drainage of collection of fluids post-operatively or instances where the tumor has been removed and some pus or blood may need to be drained externally. These are just examples.

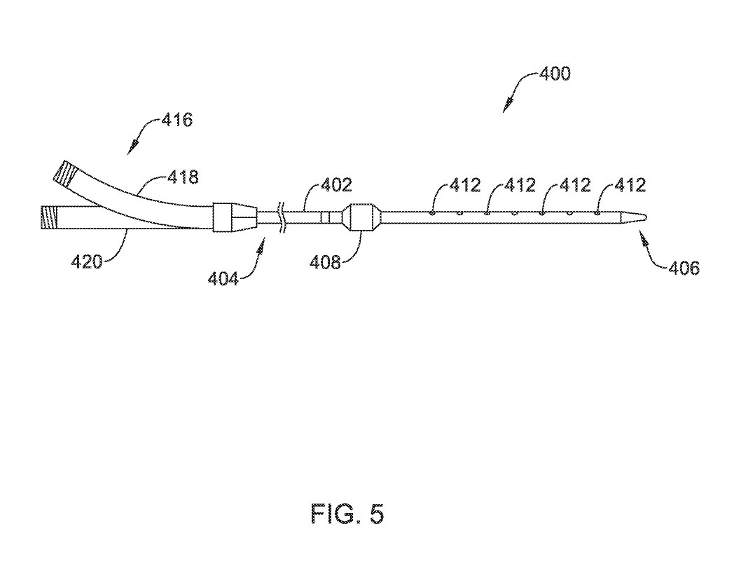

FIG. 5 illustrates another illustrative embodiment including a single balloon. The medical device 400 may include an elongate shaft 402 having a proximal end 404 and a distal end 406. While not explicitly shown, the device 400 may include a drainage lumen and an inflation lumen. The lumens of the medical device 400 may be similar in form and function to the lumens 126, 128 of the medical device 100. The proximal end 404 may be connected to a hub assembly 416 configured to remain outside the body. The hub assembly 416 may further include a drainage port 418 and a Luer valve 420. The elongate shaft 402 may include an inflatable balloon 408 positioned proximal to the distal end 406 of the elongate shaft 402. A plurality of apertures 412 may be positioned distal to the inflatable balloon 408 and in fluid communication with the drainage lumen.

Various embodiments, functionalities, and features of the elongate shaft 402, the hub assembly 416, the inflatable balloon 408 of the medical device 400, and the plurality of apertures 412 may be similar to that of the elongate shaft 102, the hub assembly 116, the inflatable balloons 108, 110, and the plurality of apertures 112, 114 of the medical device 100 respectively.

In some other embodiments, the medical device 400 may be used to drain other fluids such as bile juice, pus, blood or other fluids from the patient's body.

Portions of the medical devices 100, 400 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical devices 100, 400 may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the medical devices 100, 400 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical devices 100, 400 to achieve the same result.

In some embodiments, a degree of Magnetic Resonance Imaging (MRI) compatibility is imparted into the medical devices 100, 400. For example, the medical devices 100, 400 or portions thereof, may be made of a material that does not substantially distort the image and create substantial artifacts (i.e., gaps in the image). Certain ferromagnetic materials, for example, may not be suitable because they may create artifacts in an MRI image. The medical devices 100, 400 or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers for the medical devices 100, 400 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Those skilled in the art will recognize that the present disclosed subject matter may be manifested in a variety of forms other than the specific embodiments described and contemplated herein. Accordingly, departure in form and detail may be made without departing from the scope and spirit of the present disclosure as described in the appended.

What is claimed is:

1. A medical device comprising:
an elongate shaft having a proximal end, a distal end, a first lumen, and a second lumen;
a first inflatable balloon positioned proximal to the distal end of the elongate shaft and in fluid communication with the first lumen; and
at least one aperture positioned adjacent to the first inflatable balloon and in fluid communication with the second lumen.

2. The medical device of claim 1, wherein the at least one aperture comprises a plurality of apertures.

3. The medical device of any one of claims 1, wherein the at least one aperture is positioned distal of the inflatable balloon.

4. The medical device of any one of claims 1, wherein the at least one aperture is positioned proximal of the first inflatable balloon.

5. The medical device of any one of claims 1 further comprising a second inflatable balloon.

6. The medical device of claim 5, wherein the second inflatable balloon is positioned proximal to the first inflatable balloon.

7. The medical device of any one of claims 5, wherein the at least one aperture comprises a first plurality of apertures distal to the first inflatable balloon and a second plurality of apertures positioned proximal to the second inflatable balloon.

8. The medical device of any one of claims 1 further comprising a one-way valve in fluid communication with the first lumen.

9. The medical device of any one of claims 1, wherein the first lumen has a first cross-sectional area and the second lumen has a second cross-sectional area larger than the first cross-sectional area.

10. The medical device of any one of claims 1, further comprising a hub assembly affixed adjacent to the proximal end of the elongate shaft, the hub assembly including a Luer valve and a drainage port.

11. A medical device comprising:
an elongate shaft having a proximal end, a distal end, a first lumen, and a second lumen;
a hub assembly affixed adjacent to the proximal end, the hub assembly including a Luer valve and a drainage port;
a first inflatable balloon in fluid communication with the first lumen;
a second inflatable balloon in fluid communication with the first lumen; and
a plurality of holes in fluid communication with the second lumen.

12. The medical device of claim 11, wherein the second inflatable balloon is positioned proximal to the first inflatable balloon.

13. The medical device of claim 12, wherein at least some of the plurality of holes are positioned distal of the first inflatable balloon.

14. The medical device of any one of claims 12, wherein at least some of the plurality of holes are positioned proximal of the second inflatable balloon.

15. The medical device of any one of claims 11 further comprising a one-way valve in fluid communication with the first lumen.

16. The medical device of any one of claims 11, wherein the first lumen has a first cross-sectional area and the second lumen has a second cross-sectional area larger than the first cross-sectional area.

17. The medical device of any one of claims 11, wherein the Luer valve is in fluid communication with the first lumen and the drainage port is in fluid communication with the second lumen.

18. A medical device comprising:
an elongate shaft having a proximal end, a distal end, a first lumen, and a second lumen;
a hub assembly affixed adjacent to the proximal end, the hub assembly including a Luer valve and a drainage port;
a first inflatable balloon positioned adjacent to the distal end and in fluid communication with the first lumen;
a second inflatable balloon positioned proximal to the first inflatable balloon and in fluid communication with the first lumen;
a first plurality of holes positioned distal to the first inflatable balloon and in fluid communication with the second lumen; and
a second plurality of holes positioned proximal to the second inflatable balloon and in fluid communication with the second lumen.

19. The medical device of claim 18, wherein the first lumen has a first cross-sectional area and the second lumen has a second cross-sectional area larger than the first cross-sectional area.

20. The medical device claim 18, further comprising a one-way valve in fluid communication with the first lumen.
